SEQUENCE LISTING (1 sequence)

SEQ ID NO: 1 - protein, 280 aa, Homo sapiens
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYK

(19) United States
(12) Patent Application Publication
(10) Pub. No.: US 2024/0350512 A1
(43) Pub. Date: Oct. 24, 2024

(54) CYTOSKELETAL TENSION REGULATORS AND FAS LIGAND-COMBINATION COMPOSITIONS AND METHODS OF TREATMENT USING THE SAME

(71) Applicant: Yale University, New Haven, CT (US)

(72) Inventors: Mehmet KURAL, New Haven, CT (US); Laura NIKLASON, New Haven, CT (US)

(21) Appl. No.: 18/687,104

(22) PCT Filed: Aug. 30, 2022
(86) PCT No.: PCT/US2022/075664

§ 371 Date: Feb. 27, 2024

Related U.S. Application Data
(60) Provisional application No. 63/239,033, filed on Aug. 31, 2021.

(51) Int. Cl. A61K 31/551; A61K 31/472; A61K 31/4745; A61K 38/19; A61P 35/00; C07K 16/28

(52) U.S. Cl. CPC A61K 31/551; A61K 31/472; A61K 31/4745; A61K 38/191; A61P 35/00; C07K 16/2878; C07K 2317/75

(57) ABSTRACT

In various aspects and embodiments the invention provides a method of treating cancer in a subject in need thereof, the method comprising administering to the subject a pharmaceutical composition comprising an effective amount of a cytoskeletal tension regulator and an effective amount of a polypeptide having 80% or greater sequence identity to SEQ ID NO: 1 Fas Ligand (FasL).

### CROSS-REFERENCE TO RELATED APPLICATIONS

The present application claims priority under 35 U.S.C. § 119(e) to U.S. Provisional Patent Application No. 63/239,033, filed Aug. 31, 2021, which application is incorporated herein by reference in its entirety.

### STATEMENT REGARDING FEDERALLY SPONSORED RESEARCH OR DEVELOPMENT

This invention was made with government support under HL148819 awarded by National Institutes of Health. The government has certain rights in the invention.

### SEQUENCE LISTING

The present application contains a Sequence Listing which has been submitted in XML format via Patent Center and is hereby incorporated by reference in its entirety. Said XML file, created on Aug. 8, 2022, is named 047162-7340WO1_Sequence Listing.xml and is 2 kilobytes in size.

### BACKGROUND OF THE INVENTION

Fas (CD95) is a cell surface death receptor from the tissue necrosis factor (TNF) superfamily that forms micro-aggregates and triggers apoptosis upon binding to its ligand. There are different mechanisms that block Fas mediated apoptosis in both normal and neoplastic cells. Insufficient Fas receptor expression on the cell surface is one of the mechanisms that makes cancer cells insensitive to Fas ligand. For instance, while 99% of the normal human bronchial epithelial cells or approximately 30% of low-grade carcinoid cells (such as H720) express Fas receptors both on the cell membrane and in the cytoplasm, high grade lung tumor cells (e.g. H810, H2106, and H69) completely lose surface expression of Fas. Similarly, multiple types of glioma, esophageal adenocarcinoma, and colon cancer cells also lack the expression of Fas receptors on their surface.

Fas signaling has tumor-promoting, rather than apoptotic, effects on some resistant cancer types. Exogeneous Fas ligand and stimulation of Fas can increase motility and proliferation of chemoresistant cancer cells. Moreover, activation of Fas receptor can increase the stemness of cancer cells in some breast cancer and Fas-rich glioblastoma cells. Thus, Fas ligand can have an unexpected growth-promoting effect when used alone in some cancer cells.

Raf-1 kinase is one of the cornerstones of the Ras-Raf-MEK-ERK cascade which is highly effective in driving cell proliferation, activation, survival, and oncogenesis. In embryonic development, the role of Raf-1 is primarily to protect cells from Fas-mediated apoptosis rather than promoting proliferation by activating the (MEK)-ERK module. Extracellular stiffness and cytoskeletal tension are known to promote tumor survival and invasion. Additionally, increased cytoskeletal tension or mechanical stretch upregulates active Raf-1. There is a need in the art for compositions and methods that could be used for the treatment of cancer. The present disclosure addresses this need.

### SUMMARY OF THE INVENTION

In one aspect, the invention provides a method of treating cancer in a subject in need thereof, the method comprising administering to the subject a pharmaceutical composition comprising an effective amount of a cytoskeletal tension regulator and an effective amount of a polypeptide having 80% or greater sequence identity to SEQ ID NO: 1 Fas Ligand (FasL).

In another aspect, the invention provides a pharmaceutical composition comprising an effective amount of a cytoskeletal tension regulator and an effective amount of a polypeptide having 80% or greater sequence identity to SEQ ID NO: 1 Fas Ligand (FasL).

In various embodiments, the cytoskeletal tension regulator is selected from the group consisting of is a RhoA/Rho kinase (ROCK) inhibitors and myosin inhibitors. In various embodiments, the cytoskeletal tension regulator is selected from the group consisting of Fasudil, HA-1077 Hydrochloride, H-1152, Netarsudil, Ripasudil and Blebbistatin.

In various embodiments, the cancer is an epithelial cell cancer. In various embodiments, the cancer is triple negative breast cancer, non-small cell lung carcinoma, prostate cancer or liver cancer.

In various embodiments, the polypeptide 85% or greater, 90% or greater, 95% or greater sequence identity to SEQ ID NO: 1. In various embodiments, the polypeptide has SEQ ID NO: 1.

In various embodiments, the pharmaceutical composition is locally delivered to the cancer.

In various embodiments, the pharmaceutical composition is administered systemically.

In various embodiments, the subject is a human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1C: Fas-FasL interaction, effect of RAF-1 and intracellular tension. FIG. 1A: Following Fas ligand binding to Fas receptor, receptors form trimers and activate Rok-α. This is followed by phosphorylation of Ezrin which facilitates the formation of micro aggregates of Fas receptor trimers and initiates apoptotic cascade. Active Raf-1 can block this event by modulating Rok-α and causes Fas receptor internalization. FIG. 1B: It is known that mechanical stretch activates Raf-1. In cancer cells, increased intracellular mechanical stress might be responsible for activated Raf-1 and lack of Fas receptors on the cell surface. FIG. 1C: Reduction of intracellular tension may bring Fas receptors to the cell surface and make cancer cells sensitive to Fas ligand.

FIGS. 2A-2E: Fas receptor expression on the cancer cell surface. FIG. 2A: Addition of either Fasudil, Blebbistatin, or GW5074 to the cell culture medium for 2 hours increased the Fas receptor staining on the cancer cells, but not on HUVECs. FIG. 2B: all of the five cancer cell lines express significantly higher basal Fas expression than that of HUVECs. Fold increase of Fas staining density as response to treatment with FIG. 2C: Fasudil, FIG. 2D: Blebbisatin, and FIG. 2E: GW5074.

FIGS. 3A-3E: Fortified Fas-mediated apoptosis in cancer cells. FIG. 3A: Exogenous Fas ligand (400 ng/mL) decreased cell viability significantly in U87 cells when combined with either 40 μM Fasudil, or 10 μM Blebbistatin, or 20 μM GW5074. FIG. 3B: representative light microscopy images of U87 cells showing the effect of Fasudil-Fas ligand combination. For the other cancer cell types see FIG. 7. Fasudil-Fas ligand combination decreased the viability of A549, HepG2, SUM159, PC3, and BT549 cells synergistically (FIGS. 3C-3D). FIG. 3E: viability of HUVECs, HBEs, and iPS-Cardiomyocytes did decrease significantly with Fasudil-Fas ligand treatment, but this decrease was not dramatic as in cancer cells (****: p<0.0001, ***: p<0.005, *: p<0.05; ordinary one-way ANOVA, Tukey's multiple comparisons test).

FIGS. 4A-4H: Fortified Fas-mediated apoptosis in glioblastoma brain organoid model. FIG. 4A: Cortical brain organoids were formed by mixing hESCs with U87 cells with the percentages of 99.5% and 0.5%, respectively. FIG. 4B: when 78-day-old healthy organoids were treated with Fas ligand and Fasudil combination the ratio of the number of TUNEL⁺ cells to total number of nuclei was not significantly different than untreated group (p=0.79; unpaired t test) (FIG. 4C). Early-stage (12-day-old) cortical organoids containing RFP-labeled U87 cells were treated with 400 ng/mL Fas ligand and 40 μM Fasudil on day 12 and 15. The RFP-labeled regions decreased dramatically by day 18 (FIGS. 4D-4E). When 75-day-old organoids containing RFP-labeled U87 were treated with Fas ligand-Fasudil combination, the RFP-expressing regions almost disappeared on day-78 (white arrows in FIGS. 4F-4H) (p=0.022; unpaired t test).

FIGS. 5A-5E: Fortified Fas-mediated apoptosis in glioblastoma brain organoid model. FIGS. 5A-5B: cell types in UMAP plot of glioblastoma brain organoids. ExN: Excitatory neuron, InhN: Inhibitory neuron, IPC: Intermediate progenitor cell, RGC: Radial glia cell, NPC: Neuronal progenitor cell, MAS: Masture astrocyte, AS: Astrocyte, GPC: Glial progenitor cell, NEC: Neuroepithelial cell, UN: Unknown, GB: Glioblastoma. FIG. 5C: after 24 hours of treatment with Fas ligand and Fasudil combination, genes related to apoptotic pathways, DNA damage, and oxidative stress increased in GB cells while genes related to metabolic processes decreased. FIG. 5D: UMAP plots of glioblastoma brain organoids at t=0, 24 hours, and 72 hours of treatment with Fas ligand and Fasudil combination shows that the glioblastoma cluster (encircled in red) gets smaller and disappear by time. FIG. 5E: portions of varying cell types in the organoid at three different time points. The portion of the GB cells becomes near zero after 72 hours of treatment.

FIGS. 6A-6D: Fasudil-FasL combination treatment of U-87 glioblastoma in vivo. FIGS. 6A-6B: Tumor sizes in Fasudil-FasL combination group was significantly smaller than those in any other group (unpaired t test between PBS and Fasudil-FasL combination groups p<0.005). FIG. 6C: the survival probability in Fasudil-FasL combination group was significantly higher than any group. None of the tumors in this group reached to terminal size (1 mm³). FIG. 6D: Extracted tumors one month after the initiation. The tumor in one of mice in Fasudil-FasL combination group was completely cured.

FIG. 7: Fasudil-FasL combination treatment of A549, HepG2, SUM169, and PC3 cells in vitro. Bright field images of lung cancer cells (A549), liver cancer cells (HepG2), triple negative breast cancer cells (SUM159), and prostate cancer (PC3) shows the dramatic apoptotic effect of Fasudil-FasL combination after 48 hours of treatment.

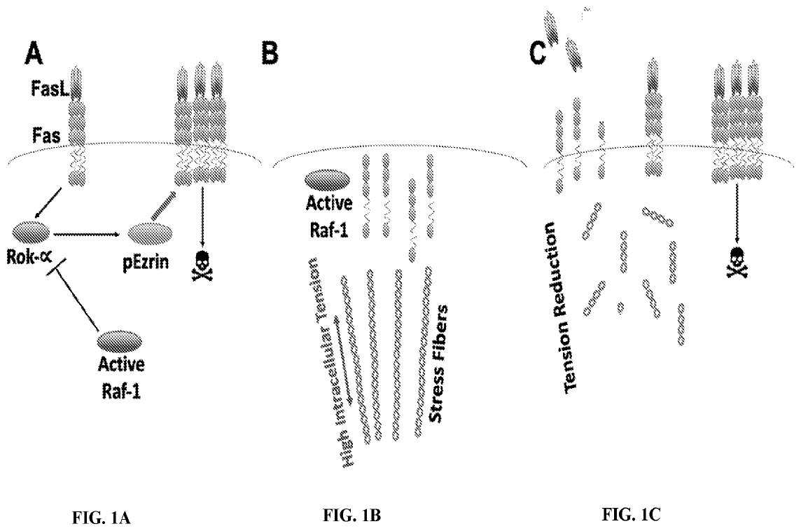

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20% or ±10%, more preferably ±5%, even more preferably ±1%, and still more preferably ±0.1% from the specified value, as such variations are appropriate to perform the disclosed methods.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, subcutaneous, oral, aerosol, parenteral, ophthalmic, pulmonary and topical administration.

As used herein, the term “cytoskeletal tension regulator” means a molecule which reduces the cytoskeletal tension of cells contacted by the molecule. In various embodiments, the cytoskeletal tension regulator is a RhoA/Rho kinase (ROCK) inhibitor. In various embodiments, the cytoskeletal tension regulator is a myosin inhibitor.

An “effective amount” or “therapeutically effective amount” of a compound is that amount of compound that is sufficient to provide a beneficial effect to the subject to which the compound is administered. An “effective amount” of a delivery vehicle is that amount sufficient to effectively bind or deliver a compound.

As used herein, the term “Fas ligand”, “FasL”, “CD95L” or “CD178” refers to the protein having the amino acid sequence SEQ ID NO: 1:
1 mqqpfnypyp qiywvdssas spwappgtvl pcptsvprrp gqrrpppppp ppplpppppp
61 pplpplplpp lkkrgnhstg lcllvmffmv lvalvglglg mfqlfhlqke laelrestsq
121 mhtasslekq ighpspppek kelrkvahlt gksnsrsmpl ewedtygivl lsgvkykkgg
181 lvinetglyf vyskvyfrgq scnnlplshk vymrnskypq dlvmmegkmm sycttgqmwa
241 rssylgavfn ltsadhlyvn vselslvnfe esqtffglyk
   for the human homolog.

As used herein, the term, “blebbistatin” refers to a compound having formula:

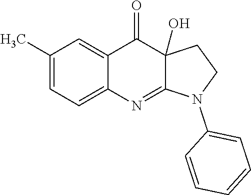 [SMILES: Cc1ccc2c(c1)C(=O)C1(O)CCN(c3ccccc3)C1=N2]

or a pharmaceutically acceptable salt thereof.

As used herein, the term, “fasudil” refers to a compound having formula:

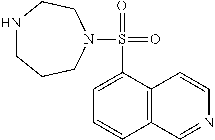 [SMILES: O=S(=O)(c1cccc2cnccc12)N1CCCNCC1]

or a pharmaceutically acceptable salt thereof.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly, the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

### Methods of Treating Cancer

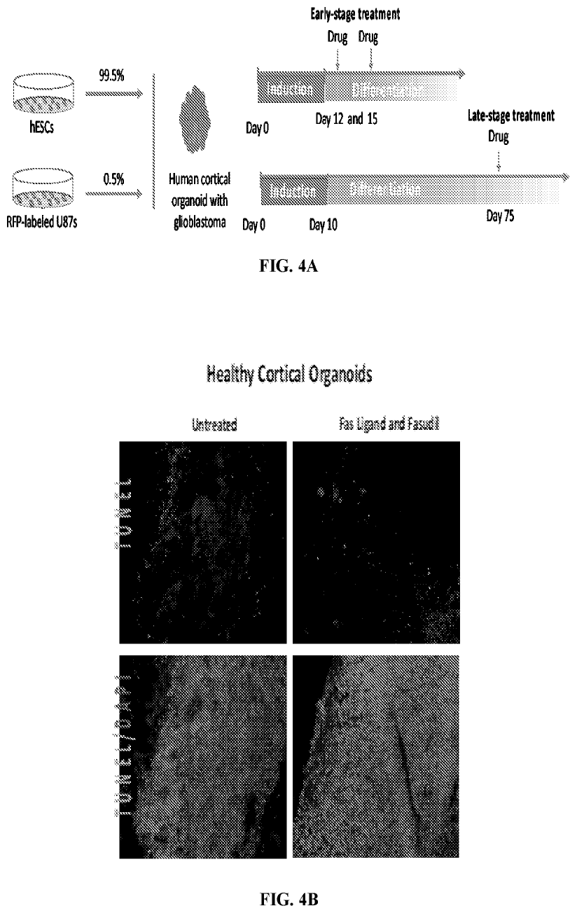

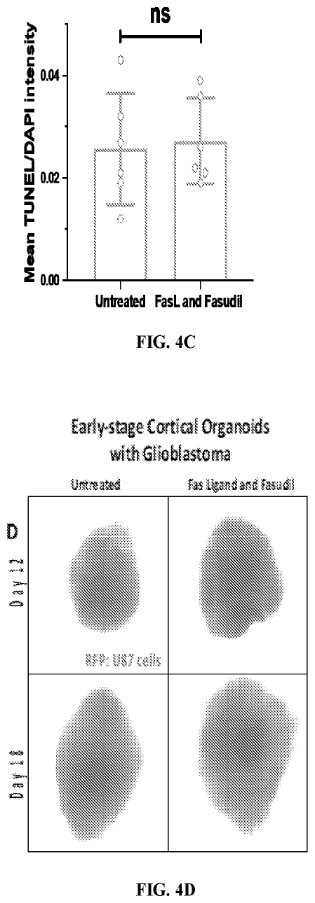

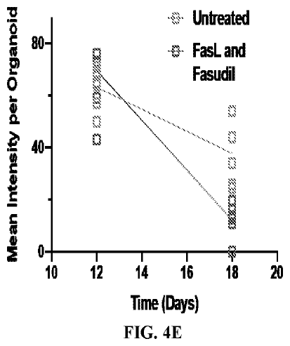

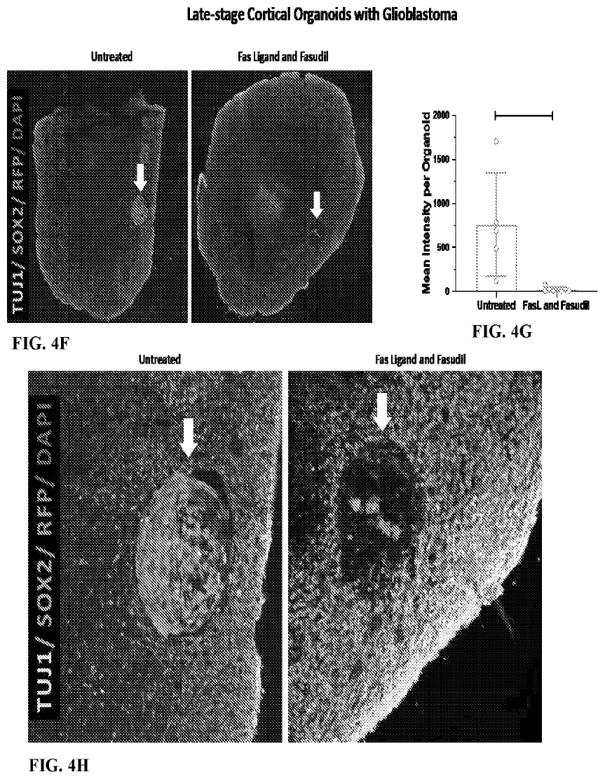

In one aspect, the invention provides a method of treating cancer in a subject in need thereof, the method comprising administering to the subject a pharmaceutical composition comprising an effective amount of a cytoskeletal tension regulator and an effective amount of a polypeptide having 80% or greater sequence identity to SEQ ID NO: 1 Fas Ligand (FasL). Without meaning to be limited by theory, it has now been discovered that the combination of a cytoskeletal tension regulator and Fas ligand has a synergistic effect in the treatment of cancer. FIGS. 4A-4E illustrate this effect with respect to the cytoskeletal tension regulators Fasudil and Blebbistatin. As previously shown, the use of engineered/enhanced Fas ligands alone as a cancer drug requires higher doses which results in serious liver toxicity and a drop in platelets in preclinical studies. Moreover, it is reported that cancer cells can develop resistance to engineered Fas ligand treatment when it is used alone. Using the methods and compositions taught herein it is possible to achieve potent and selective apoptotic effect on cancer cells while sparing non-cancer cells.

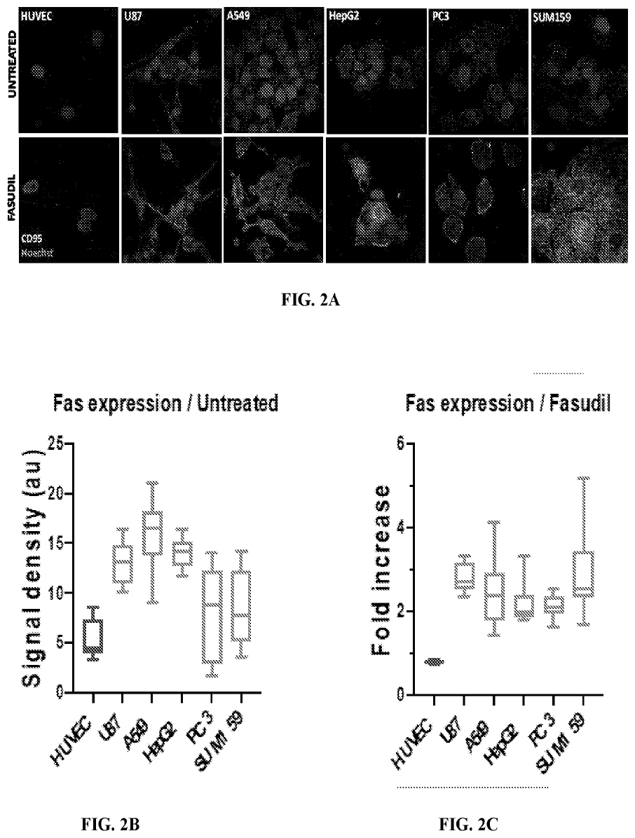

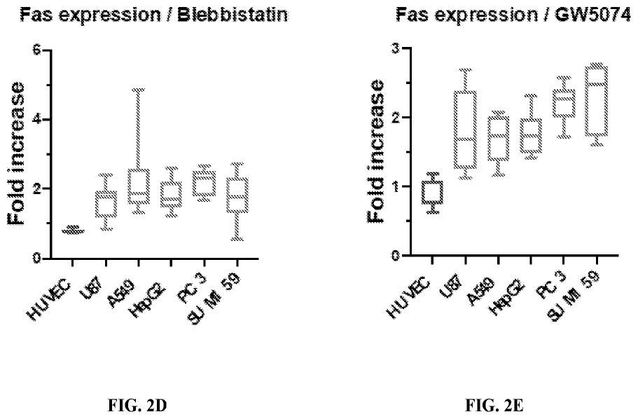

In various embodiments, the cytoskeletal tension regulator is selected from the group consisting of RhoA/Rho kinase (ROCK) inhibitors, myosin inhibitors and c-Raf inhibitors. In various embodiments, the cytoskeletal tension regulator is GW5074, Fasudil or Blebbistatin. FIG. 2 and Example 1 present data showing that the pharmaceutical composition, as described elsewhere herein, comprising the cytoskeletal tension regulator and the polypeptide is potently effective against various cancers, such as, for example, triple negative breast cancer, non-small cell lung carcinoma, prostate cancer, and liver cancer cells. A person of skill in the art will recognize that in various aspects and embodiments the methods and compositions of the invention may be applied to any solid tumor. In various embodiments, the cancer is a connective tissue cancer. In various embodiments, the cancer is an epithelial cell cancer. In various embodiments, the cancer is triple negative breast cancer, non-small cell lung carcinoma, prostate cancer or liver cancer. In various embodiments, the cancer is a glioma. In various embodiments, the cancer is glioblastoma. In various aspects and embodiments, the Fas-ligand may be a compound that causes Fas receptor oligomerization. In various embodiments, an activating Fas antibody, Turmeric or Resveratrol can be used instead og Fas ligand.

The method according to claim 1, wherein the polypeptide has 80% or greater, 85% or greater, 90% or greater, 95% or greater sequence identity to SEQ ID NO: 1. In various embodiments, the polypeptide has about or greater than about 80%, 81%, 82%, 83%, 84%, 85%, 86%, 87%, 88%, 89%, 90%, 91%, 92%, 93%, 94%, 95% sequence identity to SEQ ID NO: 1. In various embodiments, the polypeptide has SEQ ID NO: 1. A skilled person will recognize that the role of the Fas ligand or Fas antibody in the method of the invention is to trigger Fas mediated apoptosis. Accordingly, in various embodiments, the polypeptide may be an active fragment of the Fas ligand.

In various embodiments, the pharmaceutical composition is locally delivered to the cancer. In various embodiments, methods of delivery include intratumoral injections, drug-eluting biodegradable sutures or polymer implants, drug-eluting nano- or micro-particles.

In various embodiments, the subject is a mammal. In various embodiments, the subject is a human.

### Compositions

In another aspect, the invention provides a pharmaceutical composition comprising an effective amount of a cytoskeletal tension regulator and an effective amount of a polypeptide having 80% or greater sequence identity to SEQ ID NO: 1 Fas Ligand (FasL). In various embodiments, the cytoskeletal tension regulator is selected from the group consisting of RhoA/Rho kinase (ROCK) inhibitors, myosin inhibitors, and c-Raf inhibitors. In various embodiments, the cytoskeletal tension regulator is a ROCK or Rho pathway inhibitor. In various embodiments, the cytoskeletal tension regulator is Fasudil, HA-1077 Hydrochloride, H-1152, Netarsudil, Ripasudil, or Blebbistatin.

### Administration/Dosage/Formulations

The regimen of administration may affect what constitutes an effective amount. The therapeutic formulations may be administered to the subject either prior to or after the onset of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat disease in the patient. An effective amount of the therapeutic compound necessary to achieve a therapeutic effect may vary according to factors such as the state of the disease or disorder in the patient; the age, sex, and weight of the patient; and the ability of the therapeutic compound to treat disease in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of disease in a patient.

The carrier may be a solvent or dispersion medium containing, for example, water, ethanol, polyol (for example, glycerol, propylene glycol, and liquid polyethylene glycol, and the like), suitable mixtures thereof, and vegetable oils.

In certain embodiments, the compositions of the invention are administered to the patient in dosages that range from one to five times per day or more. In other embodiments, the compositions of the invention are administered to the patient in range of dosages that include, but are not limited to, once every day, every two days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physical taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of disease in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions of the invention include oral, nasal, rectal, intravaginal, parenteral, intravenous, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

The present invention also includes a multi-layer tablet comprising a layer providing for the delayed release of one or more compounds of the invention, and a further layer providing for the immediate release of a medication for treatment of certain diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a disease in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in practicing the following examples are here described:

In the present study, the effect of the ROCK inhibitor Fasudil, Myosin-II inhibitor Blebbistatin, or the Raf-1 inhibitor GW5074 on Fas receptor translocation on surface of U87, A549, HepG2, PC3, and SUM159 cells was evaluated. The effect of these drugs on cell viability when used alone or in combination with exogenous, recombinant Fas ligand was analyzed. Next, the effects of Fasudil and Fas ligand combination on glioblastoma growth and on viability of non-cancerous cells in engineered brain organoids was investigated. Finally, the in vivo efficacy of the Fasudil-Fas ligand combination on preventing tumor growth in a xenograft U87 tumor model in nude mice was tested.

### Example 1: Restoration of Fas Receptors on the Cancer Cell Surface

It was speculated that the active Raf-1, due to increased intracellular tension in cancer cell, can cause lack of Fas receptors on the cell surface and that either inhibition of Raf-1 or reduction of mechanical tension in the cytoskeleton can restore the Fas receptors on cell surface. To test this theory, U87 (glioblastoma), A549 (non-small cell lung cancer, resistant to apoptosis, FAS ligand alone increases proliferation), HepG2 (liver cancer), PC3 (prostate cancer), and SUM159 (triple negative breast cancer) cells were treated either with 20 μM Raf-1 inhibitor GW5074, 40 μM ROCK inhibitor Fasudil, or 10 μM myosin inhibitor Blebbistatin for 2 hours. The immunostaining of surface Fas receptors of cancer cells increased by 2.12-3.28 folds by Fasudil, 1.76-2.15 folds by Blebbistatin, and 1.62-2.30 by GW5074 treatment, while HUVECs showed negligible Fas staining regardless of the treatment (FIGS. 2A-2E). These results demonstrate that either the direct de-activation of Raf-1 or decreasing the intracellular mechanical tension can increase expression of the Fas receptors on these cancer cell surface but not on certain noncancer cells.

### Example 2: Effect of Soluble Fas Ligand and Tension-Reduction Combination on Cancer Cell Viability

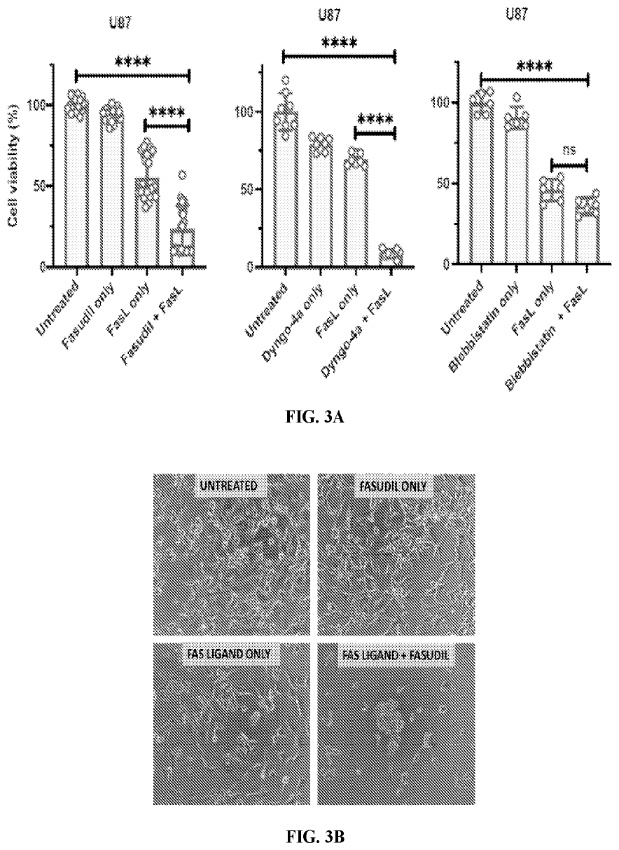

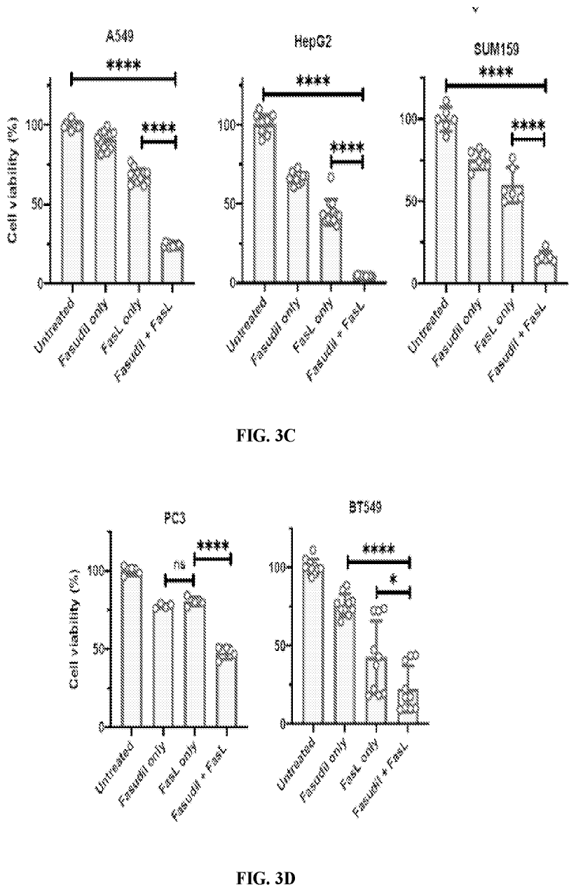

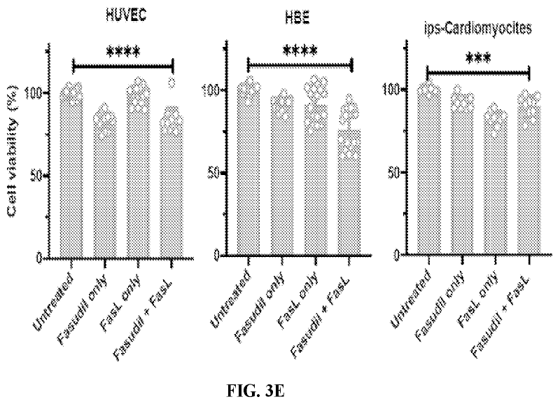

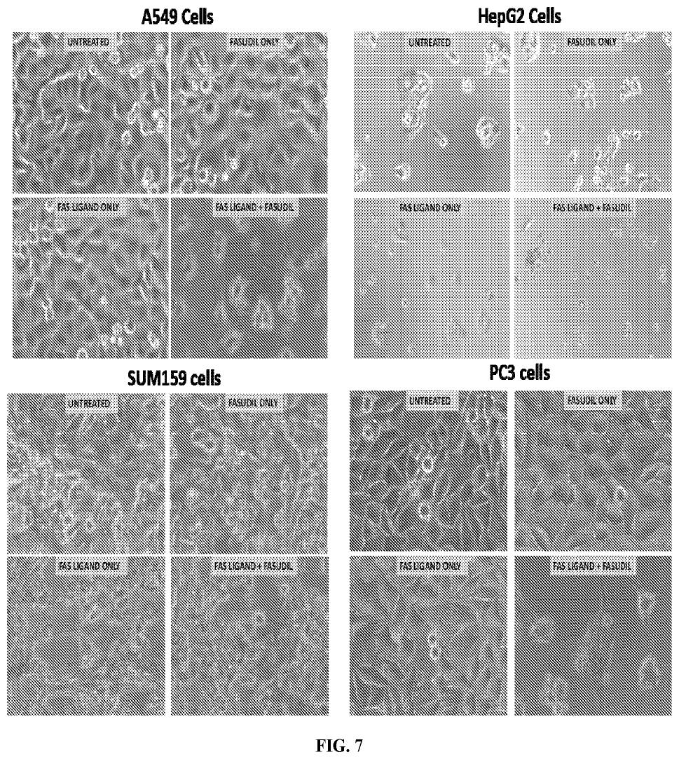

After showing that Fas receptor expression on the cell surface can be increased with different ques targeting cytoskeletal tension, the Fas-mediated apoptosis triggered by the exogenous Fas ligand when combined with Fasudil, Blebbistain, or GW5074 was studied. The cell viability by quantifying the ATP level present in the cell culture medium after 48 hours of treatment was assessed. When human recombinant Fas ligand was added to culture medium alone, it had mild to moderate effect on cell viability: 55.6%, 67.8%, 44.6%, 59.9%, %80.2, and 58.0% of the control, in U87, A549, HepG2, SUM159, PC3, and BT549 cells respectively. However, addition of either Fasudil, or Blebbistain, or GW5074 increased the effect of Fas ligand synergistically and decreased the cell viability of dramatically to 3.6-24.2%, 11.2-39.9%, and 17.2-59.1% of the control, respectively for each drug type (FIGS. 3A-3D, FIG. 7). Interestingly, viability of HUVECs, HBE cells, and iPS-derived cardiomyocytes were affected by Fasudil and Fas ligand combination relatively minimally compared to cancer cells (93.1%, 73.1%, and 83.2% of the control, in HUVECs, HBE cells, and iPS-derived cardiomyocytes respectively) (FIG. 3E). These results demonstrate that targeting either cytoskeletal tension or active Raf-1 increases the sensitivity of cancer cells to Fas ligand while sparing endothelial cells, bronchial epithelial cells or cardiomyocytes.

### Example 3: Efficacy of Fas Ligand and Fasudil Combination on Glioblastoma Model in Cortical Brain Organoids

Next, in vitro glioblastoma model based on embryonic stem cell-derived cortical brain organoids was generated to study the effect of Fas ligand and Fasudil combination on U87 cells in organoids and other cellular components (FIG. 4A). Treatment with Fas ligand and Fasudil combination did not increase the percentage of apoptotic cells in 78-day-old healthy (not containing U87 cells) brain organoids (FIGS. 4B and 4C). When U87 cell-containing brain organoids were treated twice a week with Fas ligand and Fasudil combination, the volume of the red fluorescent protein (RFP)-labeled U87 cells decreased dramatically at the end of one week in both early (12-18 days old) (FIGS. 4D and 4E) and late-stage organoids (older than 75 days) (FIGS. 4F-4H). FIG. 4G shows the shrinkage of glioblastoma mass in the organoid leaving an acellular empty space behind. Neuron progenitor marker SOX2 and differentiated neural marker TUJ1 staining is expressed on both untreated and drug-treated organoids. Here it is demonstrated that Fas ligand and Fasudil combination can kill glioblastoma cells in brain organoids selectively with minimal effect on the viability of healthy astrocytes or neuron progenitor cells.

### Example 4: Single-Cell Mapping of Fas Ligand and Fasudil-Treated Brain Organoids with Glioblastoma

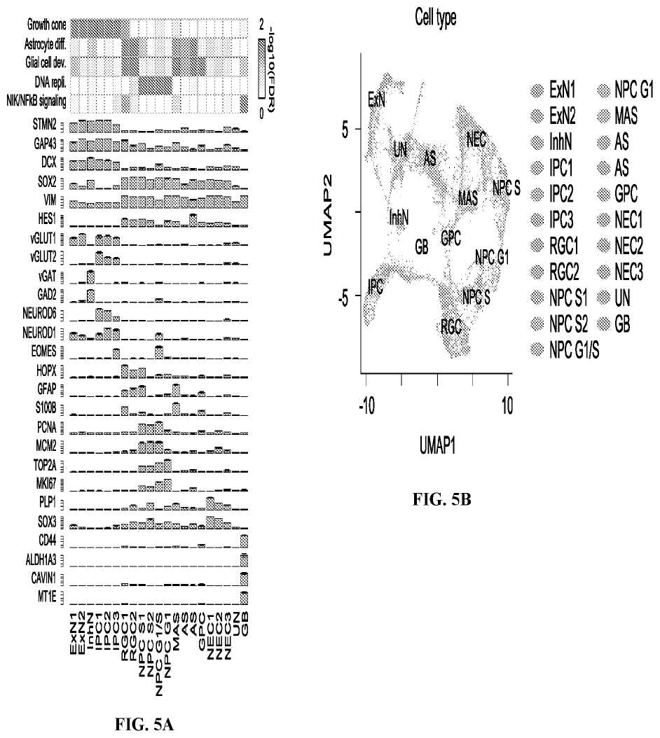

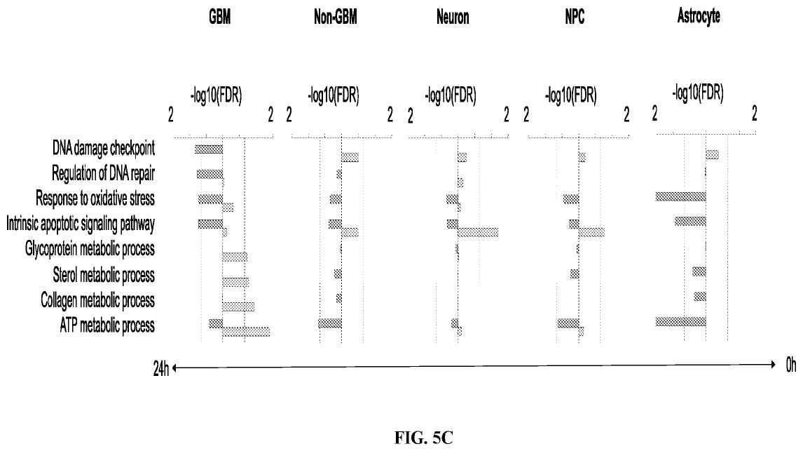

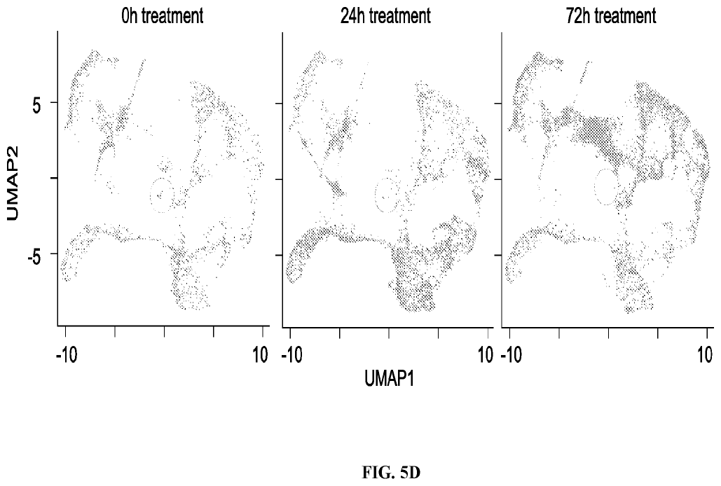

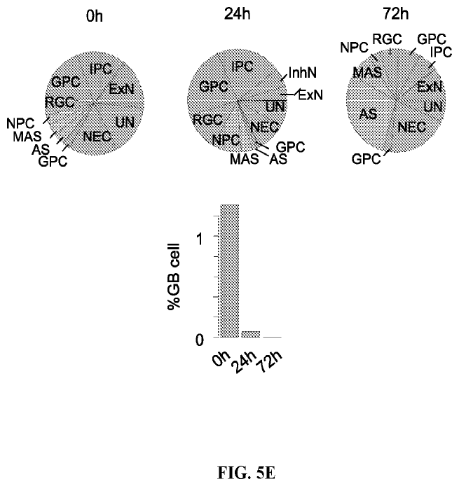

To assess the effect of Fas ligand and Fasudil combination on U87 cells in brain organoids with single cell resolution, single-cell transcriptome analysis of a total of 1391, 1097, and 2149 cells derived from organoids that are untreated, treated for 24 hours, and 72 hours, respectively, was used. UMAP plots show that after 24 hours of treatment number of U87 cells decreased dramatically and at 72 hours number of glioblastoma cells (U87) became negligible (FIGS. 5A, 5C, and 5D). Global comparison of transcriptome between Fas ligand and Fasudil combination- and non-treated organoids identified that 611 and 252 genes are significantly up- and down-regulated by the drug treatment, respectively. Gene Ontology analysis identified that the up-regulated genes are involved in oxidative stress response (e.g., GCLM, HSF1, and OGG1) and apoptosis (e.g., MSH6, DNAJA1, and TRAP1) and DNA repair (e.g., FOXO4, MDM4, and CUL4A) (FDR<0.05 by hypergeometric test). In contrast, metabolic genes for glycoprotein (e.g., MVD, OST4), ATP (e.g., COX5B, COX6C), sterol (e.g., NPC1, NPC2) and collagen (e.g., MMP2, VIM) are significantly downregulated (FDR<0.05) (FIG. 5B). However, with treatment, the upregulation of cell death-related genes and down regulation of metabolic genes were much stronger in glioblastoma cells (U87) than those in non-cancer cells as a whole, neuron, and neuron progenitor cells. Interestingly, both apoptotic genes and metabolic genes upregulated in astrocytes with the treatment.

### Example 5: Treatment of U-87 Glioblastoma Xenograft Tumors with Fas Ligand and Fasudil Combination In Vivo

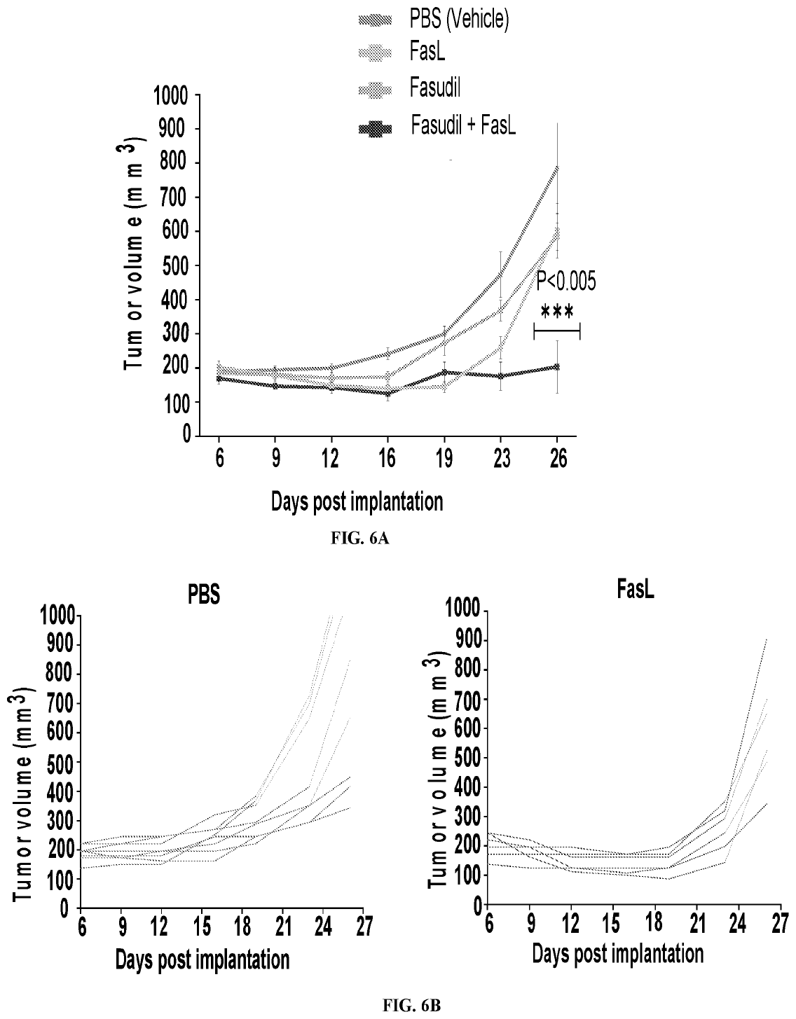

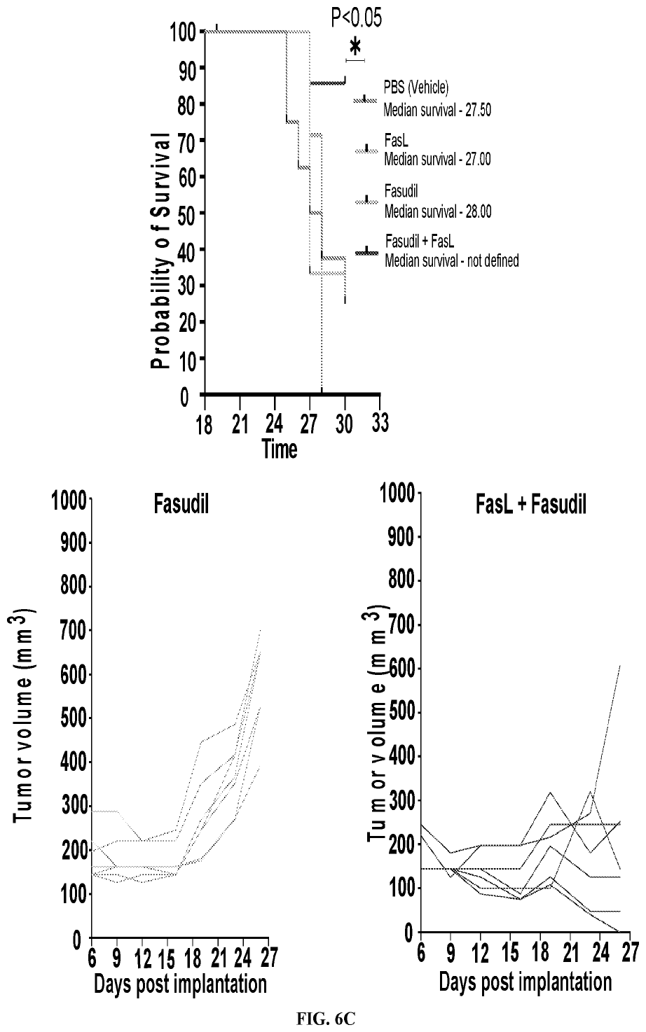

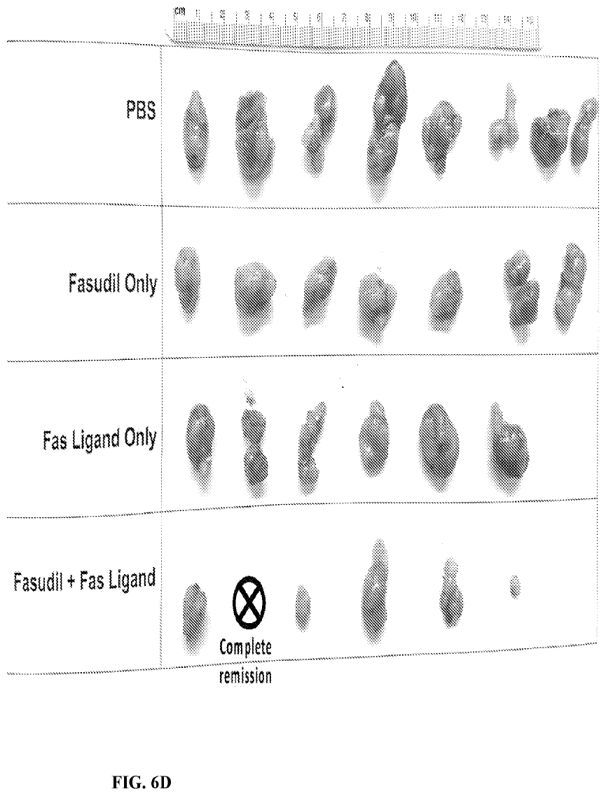

Further, the anti-tumor efficacies of Fas ligand and Fasudil combination using a U87 xenograft tumor model in nude mice were evaluated. Fas ligand and Fasudil were administrated by intraperitoneal injection when the subcutaneous tumor grew to palpable size >50 mm³ twice a week for four weeks. The mice were primed with either Fasudil or saline 24 hours before the injection of Fasudil and Fas ligand together. Only Fasudil, only Fas ligand, and only saline injections were made in the control groups. 7-10 mice with tumor volumes ranging from 50-200 mm³ were randomly assigned to each treatment arm. Terminal point for the tumor growth was determined as 1000 mm³. One month after the tumor initiation, the average tumor sizes were 809.2±373.3, 602.2±195.7, 584.4±113.8, and 235.6±219.5 mm3 in saline, FasL only, Fasudil only, and Fasudil-FasL combination groups, respectively (FIGS. 6A and 6B). The median survival probabilities were 27.5%, 27%, and 28% in saline, FasL only, and Fasudil only groups (FIG. 6C). One mouse died in Fasudil-FasL combination group due to non-tumor growth related cause. None of the mice reached terminal point in Fasudil-FasL combination group and the tumor in one of the mice became unmeasurably small (complete remission) after the fifth injection of the Fasudil-FasL combination (FIG. 6D). These results demonstrates that local administration of Fasudil-FasL combination, but not Fasudil or FasL alone, has a significant tumor suppressing effect in vivo.

### Enumerated Embodiments

The following enumerated embodiments are provided, the numbering of which is not to be construed as designating levels of importance.

Embodiment 1 provides a method of treating cancer in a subject in need thereof, the method comprising administering to the subject a pharmaceutical composition comprising an effective amount of a cytoskeletal tension regulator and an effective amount of a polypeptide having 80% or greater sequence identity to SEQ ID NO: 1 Fas Ligand (FasL).

Embodiment 2 provides the method of embodiment 1, wherein the cytoskeletal tension regulator is selected from the group consisting of a RhoA/Rho kinase (ROCK) inhibitors and myosin inhibitors.

Embodiment 3 provides the method of embodiments 1-2, wherein the cytoskeletal tension regulator is selected from the group consisting of Fasudil, HA-1077 Hydrochloride, H-1152, Netarsudil, Ripasudil and Blebbistatin.

Embodiment 4 provides the method of embodiments 1-3, wherein the cancer is an epithelial cell cancer.

Embodiment 5 provides the method of embodiments 1-4, wherein the cancer is triple negative breast cancer, non-small cell lung carcinoma, prostate cancer or liver cancer.

Embodiment 6 provides the method of embodiments 1-5, wherein the polypeptide has 80% or greater, 85% or greater, 90% or greater, 95% or greater sequence identity to SEQ ID NO: 1.

Embodiment 7 provides the method of embodiments 1-6, wherein the polypeptide has SEQ ID NO: 1.

Embodiment 8 provides the method of embodiments 1-7, wherein the pharmaceutical composition is locally delivered to the cancer.

Embodiment 9 provides the method of embodiments, 1-8, wherein the pharmaceutical composition is administered systemically.

Embodiment 10 provides the method of embodiments 1-9, wherein the subject is a human.

Embodiment 11 provides a pharmaceutical composition comprising an effective amount of a cytoskeletal tension regulator and an effective amount of a polypeptide having 80% or greater sequence identity to SEQ ID NO: 1 Fas Ligand (FasL).

Embodiment 12 provides the composition of embodiment 11, wherein the cytoskeletal tension regulator is selected from the group consisting of is a RhoA/Rho kinase (ROCK) inhibitors and myosin inhibitors.

Embodiment 13 provides the composition of embodiments 11-12, wherein the cytoskeletal tension regulator is selected from the group consisting Fasudil, HA-1077 Hydrochloride, H-1152, Netarsudil, Ripasudil and Blebbistatin.

Embodiment 14 provides the composition of embodiments 11-13, wherein the polypeptide has 85% or greater, 90% or greater, 95% or greater sequence identity to SEQ ID NO: 1.

Embodiment 15 provides the composition of embodiments 11-14, wherein the polypeptide has SEQ ID NO: 1.

Embodiment 16 provides the composition of embodiments 11-15, wherein the pharmaceutical composition is locally delivered to the cancer.

Embodiment 17 provides the composition of embodiments 11-16, wherein the pharmaceutical composition is administered systemically

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating cancer in a subject in need thereof, the method comprising administering to the subject a pharmaceutical composition comprising an effective amount of a cytoskeletal tension regulator and an effective amount of a polypeptide having 80% or greater sequence identity to SEQ ID NO: 1 Fas Ligand (FasL) or an activating Fas antibody.

2. The method according to claim 1, wherein the cytoskeletal tension regulator is selected from the group consisting of a RhoA/Rho kinase (ROCK) inhibitors and myosin inhibitors.

3. The method according to claim 1, wherein the cytoskeletal tension regulator is selected from the group consisting of Fasudil, HA-1077 Hydrochloride, H-1152, Netarsudil, Ripasudil and Blebbistatin.

4. The method according to claim 1, wherein the cancer is an epithelial cell cancer.

5. The method according to claim 1, wherein the cancer is triple negative breast cancer, non-small cell lung carcinoma, prostate cancer or liver cancer.

6. The method according to claim 1, wherein the polypeptide has 85% or greater, 90% or greater, 95% or greater sequence identity to SEQ ID NO: 1.

7. The method according to claim 1, wherein the polypeptide has SEQ ID NO: 1.

8. The method according to the claim 1, wherein the pharmaceutical composition is locally delivered to the cancer.

9. The method according to the claim 1, wherein the pharmaceutical composition is administered systemically.

10. The method according to the claim 1, wherein the subject is a human.

11. A pharmaceutical composition comprising an effective amount of a cytoskeletal tension regulator and an effective amount of a polypeptide having 80% or greater sequence identity to SEQ ID NO: 1 Fas Ligand (FasL).

12. The composition according to claim 10, wherein the cytoskeletal tension regulator is selected from the group consisting of is a RhoA/Rho kinase (ROCK) inhibitors and myosin inhibitors.

13. The composition according to claim 10, wherein the cytoskeletal tension regulator is selected from the group consisting Fasudil, HA-1077 Hydrochloride, H-1152, Netarsudil, Ripasudil and Blebbistatin.

14. The composition according to claim 10, wherein the polypeptide has 85% or greater, 90% or greater, 95% or greater sequence identity to SEQ ID NO: 1.

15. The composition according to claim 10, wherein the polypeptide has SEQ ID NO: 1.

16. The composition according to the claim 10, wherein the pharmaceutical composition is locally delivered to the cancer.

17. The composition according to the claim 10, wherein the pharmaceutical composition is administered systemically.
